(19) United States
(12) Patent Application Publication
(10) Pub. No.: US 2025/0269179 A1
(43) Pub. Date: Aug. 28, 2025

(54) NEEDLE TIP FOR INDUCING SKIN TWISTING AND PATCH THEREFOR

(71) Applicants: Suk Bae Seo, Seoul (KR); Ja Young Kim, Seoul (KR); Ho Joon Seo, Seoul (KR)

(72) Inventors: Suk Bae SEO, Seoul (KR); Ja Young Kim, Seoul (KR); Ho Joon Seo, Seoul (KR)

(73) Assignees: Suk Bae Seo, Seoul (KR); Ja Young Kim, Seoul (KR); Ho Joon Seo, Seoul (KR)

(21) Appl. No.: 19/201,618

(22) Filed: May 7, 2025

Related U.S. Application Data
(63) Continuation of application No. PCT/KR2023/017556, filed on Nov. 3, 2023.

(30) Foreign Application Priority Data
Nov. 7, 2022 (KR) 10-2022-0146724
Nov. 1, 2023 (KR) 10-2023-0148840

(51) Int. Cl. A61N 1/36; A61N 1/32

(52) U.S. Cl. CPC A61N 1/36017; A61N 1/328

(57) ABSTRACT

The present invention relates to a needle tip for inducing skin twisting, comprising: a tip portion comprising a holder and multiple needles retained on the holder so as to be inserted into skin such that multiple injection holes are formed; a high-frequency applicator for applying high-frequency waves to the multiple needles; and a rotator for rotating the tip portion.

### CROSS-REFERENCE TO RELATED APPLICATIONS

The present application is a continuation of International Patent Application No. PCT/KR2023/017556, filed on Nov. 3, 2023, which is based upon and claims the benefit of priority to Korean Patent Application Nos. 10-2022-0146724 filed on Nov. 7, 2022 and 10-2023-0148840 filed on Nov. 1, 2023. The disclosures of the above-listed applications are hereby incorporated by reference herein in their entirety.

### BACKGROUND

Embodiments of the present disclosure described herein a needle tip and a patch for inducing twisting of skin.

The skin includes an epidermal layer, a dermal layer, and a subcutaneous fat layer. The epidermal layer serves to prevent invasion of bacteria and other things and to prevent water in the body from being discharged to the outside. The dermal layer is located under the epidermal layer, includes collagen, elastic fibers, and substrates, and serves to participate in the elasticity, gloss, and tension of the skin. Furthermore, the subcutaneous fat layer is located under the dermal layer, and functions to absorb a pressure or a shock from the outside and maintain the body temperature.

As aging progresses, the skin gradually loses elasticity and wrinkles occur because, in general, the amounts of collagen, elastic fibers, and substrates in the dermis layer decrease and the amount of moisture decreases.

To solve this problem, a needle tip that stimulates the tissues that form skin by inserting a plurality of needles into the skin and at the same time outputs heat energy, such as a radio frequency (RF), to the skin to induce coagulation necrosis of the skin, thereby regenerating and activating collagen and elastic fibers in the skin has been developed.

However, the conventional needle tip cannot stimulate connection signals between cells of the skin as only a high frequency is applied to the skin.

### SUMMARY

Embodiments of the present disclosure provide a needle tip and a patch for inducing twisting of skin, by which twisting of the skin is induced while a high frequency is applied to the skin so that intercellular connection signals of the skin are stimulated through stretching of skin tissues, collagen and elastic fibers of the skin are increased, and skin regeneration may be improved.

Problems to be solved by the present disclosure are not limited to the problems mentioned above, and other problems not mentioned will be clearly understood by those skilled in the art from the following description.

According to an embodiment of the present disclosure, a needle tip for inducing twisting of skin may include a tip part including a holder, and a plurality of needles inserted into the skin to form a plurality of injection holes, a high-frequency application part that applies a high frequency to the plurality of needles, and a rotation part that rotates the tip part.

Furthermore, the rotation part may rotate the tip part while the plurality of needles are inserted into the skin.

Furthermore, the rotation part may rotate the tip part after the plurality of needles are inserted into the skin.

Furthermore, the rotation part may rotate the tip part in one direction while the plurality of needles are inserted into the skin, and then may rotate the tip part in an opposite direction.

Furthermore, the needle tip may further include a cylinder having an interior space, in which the tip part is fixed, and closely attached to the skin, and a pressure control part that forms a negative pressure or a positive pressure in the interior space.

Furthermore, the pressure control part may form the negative pressure in the interior space such that the plurality of needles are inserted into the skin being pulled into the interior space.

Furthermore, the pressure control part may form the positive pressure in the interior space when the plurality of needles are discharged from the plurality of injection holes to inject drug applied to the skin into the injection holes.

Furthermore, the high-frequency application part may apply any one of a monopolar type high frequency and a bipolar type high frequency to the plurality of needles when the tip part is rotated in one direction, and apply the other one of the monopolar type high frequency and the bipolar type high frequency to the plurality of needles when the tip part is rotated in an opposite direction.

According to an embodiment of the present disclosure, a patch for inducing twisting of skin may include an attachment pad closely attached to the skin, a high-frequency application part that applies a high frequency to the attachment pad, and a rotation part that rotates the attachment pad.

Furthermore, the rotation part may rotate the attachment pad in one direction while the attachment pad is closely attached to the skin, and then, may rotates the attachment pad in an opposite direction.

Furthermore, the patch may further include a cylinder having an interior space, in which the attachment pad is fixed, and closely attached to the skin, and a pressure control part that forms a negative pressure in the interior space, and the pressure control part may form the negative pressure in the interior space such that the attachment pad is closely attached to the skin being pulled into the interior space.

Other details of the present disclosure are included in the detailed description and the drawings.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1 is a schematic view illustrating a needle tip for inducing twisting of skin according to an embodiment of the present disclosure;

FIG. 2 is a schematic view illustrating an example of an operation process of a needle tip for inducing twisting of skin according to an embodiment of the present disclosure;

FIGS. 3 to 4 are schematic views illustrating another example of an operation process of a needle tip for inducing twisting of skin according to an embodiment of the present disclosure;

FIG. 5 is a schematic view illustrating a needle tip for inducing twisting of skin according to another embodiment of the present disclosure;

FIGS. 6 to 7 are schematic views illustrating an operation process of a needle tip for inducing twisting of skin according to another embodiment of the present disclosure;

FIG. 8 is a schematic view illustrating a patch for inducing twisting of skin according to an embodiment of the present disclosure; and

FIGS. 9 to 10 are schematic views illustrating an operation process of a patch for inducing twisting of skin according to an embodiment of the present disclosure.

### DETAILED DESCRIPTION

The above and other aspects, features and advantages of the present disclosure will become apparent from embodiments to be described in detail in conjunction with the accompanying drawings. The present disclosure, however, may be embodied in various different forms, and should not be construed as being limited only to the illustrated embodiments. Rather, these embodiments are provided as examples so that the present disclosure will be thorough and complete, and will fully convey the scope of the present disclosure to those skilled in the art. The present disclosure may be defined by the scope of the claims.

The terms used herein are provided to describe embodiments, not intended to limit the present disclosure. In the specification, the singular forms include plural forms unless particularly mentioned. The terms “comprises” and/or “comprising” used herein do not exclude the presence or addition of one or more other components, in addition to the aforementioned components. The same reference numerals denote the same components throughout the specification, and the term “and/or” includes each of the mentioned components and all combinations of one or more of the components. Hereinafter, embodiments of the present disclosure will be described in detail with reference to accompanying drawings.

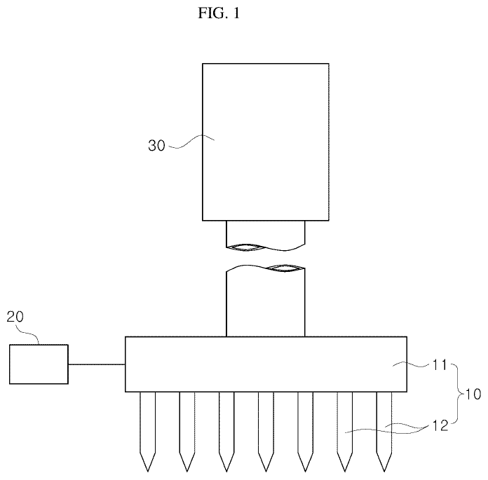

FIG. 1 is a schematic view illustrating a needle tip for inducing twisting of skin according to an embodiment of the present disclosure.

As illustrated in FIG. 1, the needle tip for inducing twisting of skin according to an embodiment of the present disclosure includes a tip part 10, a high-frequency application part 20, and a rotation part 30.

The tip part 10 serves to stimulate skin. The tip part 10 may include a holder 11 and a plurality of needles 12.

The holder 11 serves to fix the plurality of needles 12. As an example, the holder 11 may have a disk shape, and a lower surface of the holder 11 may have a circular shape so that the plurality of needles 12 are fixed in an array form. As another example, the holder 11 may have a rectangular shape, and the lower surface of the holder 11 may have a plate shape so that the plurality of needles 12 are fixed in the array form.

The plurality of needles 12 may be fixed to the holder 11 and be inserted into the skin to form a plurality of injection holes. Here, that plurality of injection holes are holes that are formed in the skin as the plurality of needles 12 are inserted into the skin. Meanwhile, drug that is applied to the skin may be injected into the plurality of injection holes, and details thereof will be described later.

As an example, the plurality of needles 12 may be fixed to the lower surface of the holder 11 in the array form.

As another example, when the plurality of needles 12 are inserted into the skin, some of the plurality of needles 12, which are disposed at the center of the holder 11, may be inserted into the skin in a more downwardly deflected state than some of the plurality of needles 12, which are disposed at a periphery of the holder 11.

Although not illustrated in the drawings, to prevent this, some of the plurality of needles 12, which are disposed at a periphery of the holder 11, may have a length that is smaller than some of the plurality of needles 12, which are disposed at the center of the holder 11. That is, some of the plurality of needles 12, which are disposed at the center of the holder 11, protrude further from the holder 11 than some of the plurality of needles, which are disposed at the periphery of the holder 11.

Meanwhile, the tip part 10 may be moved in a direction that becomes closer to the skin by a manipulation by the user or by an actuator.

The high-frequency application part 20 may serve to apply a high frequency to the plurality of needles 12. For example, the high-frequency application part 20 may apply a high frequency to the plurality of needles 12 while the plurality of needles 12 are inserted into the skin. As a result, the high frequency applied from the high-frequency application part 20 may be delivered to the skin through the plurality of needles 12 inserted into the skin.

As an example, the high-frequency application part 20 may apply a monopolar type high frequency to the plurality of needles 12 when the tip part 10 is rotated in one direction, and apply a bipolar type high frequency to the plurality of needles 12 when the tip part 10 is rotated in an opposite direction.

As another example, the high-frequency application part 20 may apply a bipolar type high frequency to the plurality of needles 12 when the tip part 10 is rotated in one direction, and apply a monopolar type high frequency to the plurality of needles 12 when the tip part 10 is rotated in an opposite direction.

As an example, when the high-frequency application part 20 applies a monopolar type high frequency to the plurality of needles 12, the high-frequency application part 20 may apply a current of the same polarity (e.g., a positive polarity) to the plurality of needles 12, and a current of an opposite polarity (e.g., a negative polarity) may be applied to a counter electrode plate (not illustrated) provided on the skin.

As another example, when the high-frequency application part 20 applies a bipolar type high frequency to the plurality of needles 12, the high-frequency application part 20 may apply a positive current to one of two adjacent needles 12 and may apply a negative current to the other one of two adjacent needles 12.

The rotation part 30 may serve to rotate the tip part 10. For example, the rotation part 30 may rotate the tip part 10 while the plurality of needles 12 are inserted into the skin. As a result, as the plurality of needles 12 inserted into the skin induce twisting of the skin while being rotated, the intercellular connection signals of the skin are stimulated through stretching of the skin tissues, collagen and elastic fibers of the skin are increased, and skin regeneration may be improved.

As an example, the rotation part 30 may be a driving motor. The driving motor may rotate the tip part 10 under the control of a processor that is manipulated by the user.

As another example, the rotation part 30 may be a knob, and the knob may manually rotate the tip part 10 according to the manipulation by the user.

Meanwhile, the rotation part 30 may rotate the tip part 10 while the plurality of needles 12 are inserted into the skin. As a result, the plurality of needles 12 are rotated while being inserted into the skin, and sequential twisting may be induced from a surface of the skin to an inside of the skin.

Furthermore, the rotation part 30 may rotate the tip part 10 after the plurality of needles 12 are inserted into the skin. As a result, the plurality of needles 12 inserted into the skin may induce the twisting of the skin while being rotated. Here, after the plurality of needles 12 are inserted into the skin, the plurality of needles 12 may be inserted into the skin to a preset depth and then may be stopped. Furthermore, after the plurality of needles 12 are inserted into the skin, the plurality of needles 12 may be fully inserted into the skin.

Furthermore, the rotation part 30 may rotate the tip part 10 in one direction while the plurality of needles 12 are inserted into the skin, and then rotate the tip part 10 in an opposite direction. As a result, the plurality of needles 12 inserted into the skin are rotated sequentially in one direction and an opposite direction, and the twisting of the skin in one direction and the twisting of the skin in an opposite direction may be sequentially induced.

A microcomputer or a programmable logic controller (PLC) may be used as the processor (not illustrated), and may serve to control the high-frequency application part 20 and the rotation part 30. As an example, the processor may include a proximity sensor that is provided in the holder 11. The proximity sensor may sense whether the plurality of needles 12 are inserted into the skin and the depth, at which the plurality of needles 12 are inserted into the skin, by measuring distances between the plurality of needles 12 and the skin. Furthermore, the processor may identify whether the plurality of needles 12 are inserted into the skin and the depth, at which the plurality of needles 12 are inserted into the skin, according to the sensing by the proximity sensor. Furthermore, the processor may operate the high-frequency application part 20 and the rotation part 30 when it is identified that the plurality of needles 12 are inserted into the skin according to the sensing by the proximity sensor.

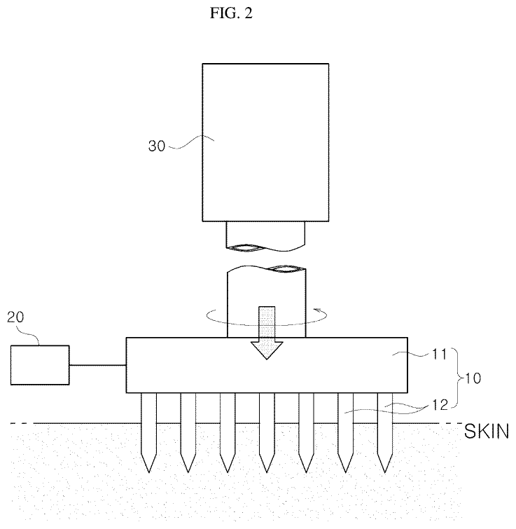

FIG. 2 is a schematic view illustrating an example of an operation process of a needle tip for inducing twisting of skin according to an embodiment of the present disclosure.

Referring to FIG. 2, an example of an operation process of a needle tip for inducing twisting of skin according to an embodiment of the present disclosure will be described. The following process may be performed by the processor.

First, the plurality of needles 12 of the tip part 10 are inserted into the skin. Here, the tip part 10 may be moved in a direction that becomes closer to the skin by a manipulation by the user, and the tip part 10 may be moved in a direction that becomes closer to the skin by an actuator.

Next, the high-frequency application part 20 applies a high frequency to the plurality of needles 12, and the high frequency is delivered to the skin through the plurality of needles 12.

At the same time, while the plurality of needles 12 are inserted into the skin, the rotation part 30 rotates the tip part 10. As a result, the plurality of needles 12 are rotated while being inserted into the skin, and sequential twisting may be induced from a surface of the skin to an inside of the skin.

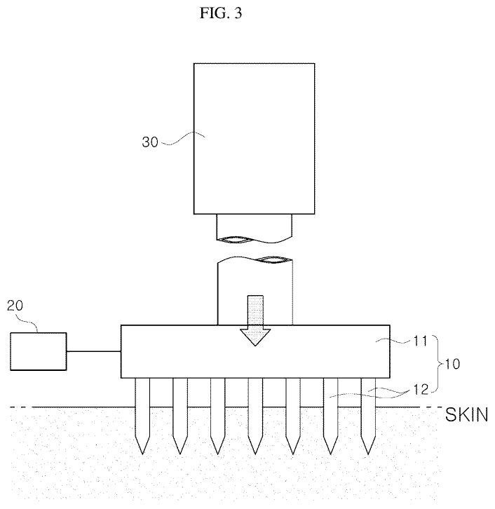

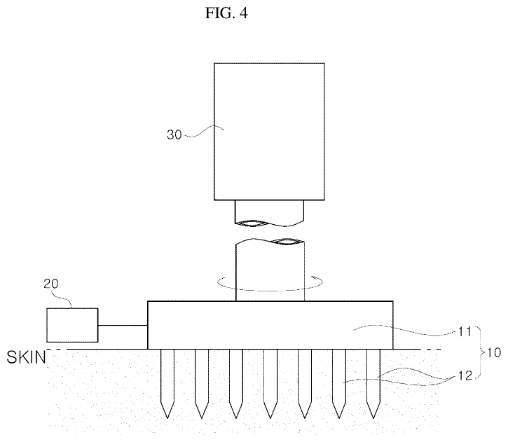

FIGS. 3 to 4 are schematic views illustrating another example of an operation process of a needle tip for inducing twisting of skin according to an embodiment of the present disclosure.

Referring to FIGS. 3 and 4, another example of an operation process of a needle tip for inducing twisting of skin according to an embodiment of the present disclosure will be described. The following process may be performed by the processor.

First, the plurality of needles 12 of the tip part 10 are inserted into the skin.

Next, the high-frequency application part 20 applies a high frequency to the plurality of needles 12, and the high frequency is delivered to the skin through the plurality of needles 12.

Subsequently, after the plurality of needles 12 are inserted into the skin, the rotation part 30 may rotate the tip part 10. As a result, the plurality of needles 12 inserted into the skin may induce the twisting of the skin while being rotated.

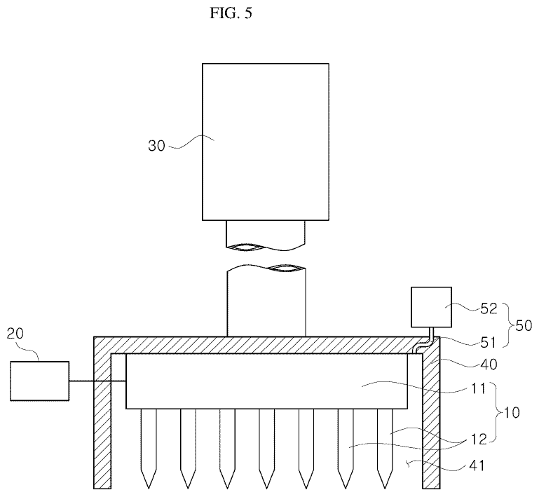

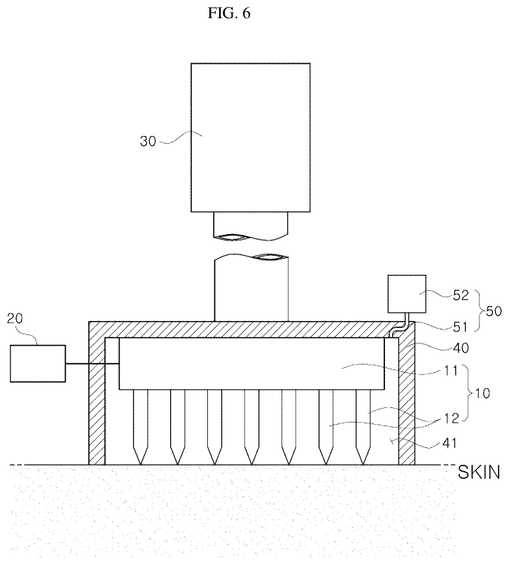

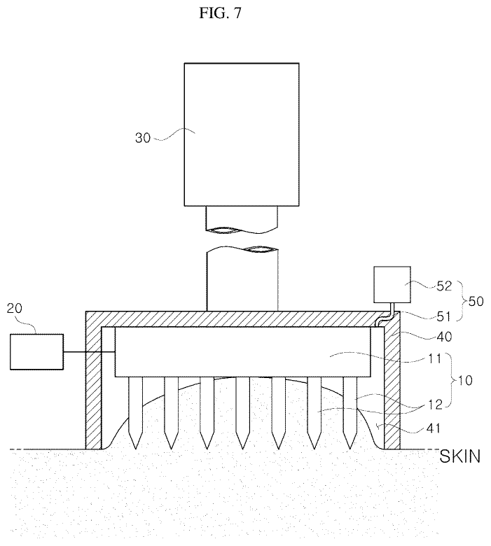

FIG. 5 is a schematic view illustrating a needle tip for inducing twisting of skin according to another embodiment of the present disclosure, and FIGS. 6 to 7 are schematic views illustrating an operation process of a needle tip for inducing twisting of skin according to another embodiment of the present disclosure.

As illustrated in FIG. 5, unlike the above embodiment, the needle tip for inducing twisting of skin according to another embodiment of the present disclosure may further include a cylinder 40 and a pressure control part 50.

The cylinder 40 has an interior space 41, to which the tip part 10 is fixed, and may be closely attached to the skin. As an example, a lower surface of the cylinder 40 may be opened and an upper surface and a circumferential surface thereof may be closed, and an interior space 41 may be formed in an interior thereof. Furthermore, the holder 11 of the tip part 10 may be fixed to an upper surface of the cylinder 40. Furthermore, the rotation part 30 may rotate the tip part 10 by rotating the cylinder 40.

The pressure control part 50 may form a negative pressure or positive pressure in the interior space 41 of the cylinder 40. Here, the negative pressure may be a pressure that is lower than the atmospheric pressure, and the positive pressure may be a pressure that is higher than the atmospheric pressure.

As an example, the pressure control part 50 may form a negative pressure in the interior space 41 of the cylinder 40 such that the plurality of needles 12 are inserted into the skin that is pulled into the interior space 41 of the cylinder 40. In this case, the plurality of needles 12 inserted into the skin may form a plurality of injection holes in the skin (see FIG. 6). Thereafter, when the plurality of needles 12 are discharged from the plurality of injection holes, the pressure control part 50 may form a positive pressure in the interior space of the cylinder 40 and inject the drug applied to the skin into the injection hole (see FIG. 7).

The pressure control part 50 may include a pressure pipe 51 that is connected to the inner space of the cylinder 40, and a pressure regulator 52 that forms a negative pressure or positive pressure in the pressure pipe 51.

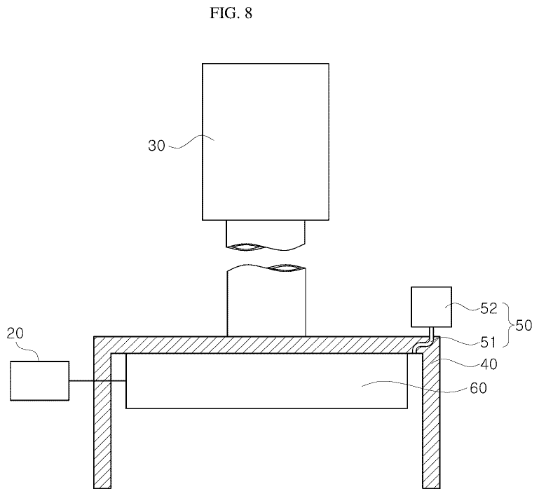

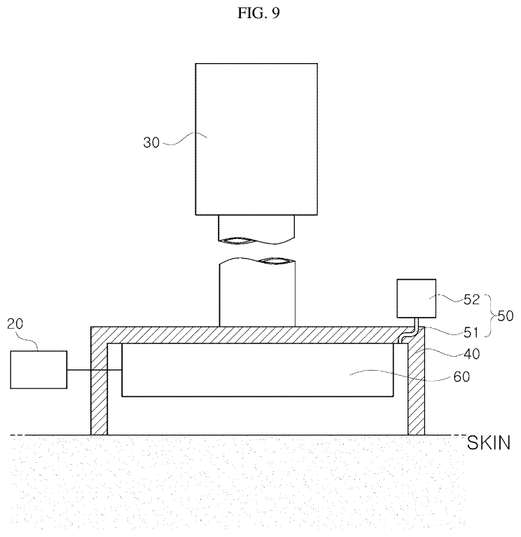

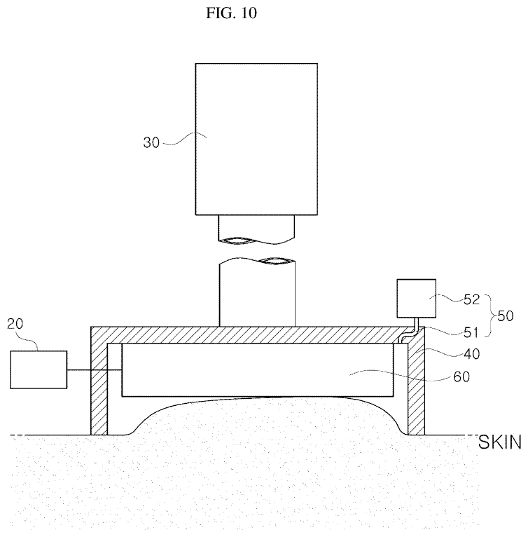

FIG. 8 is a schematic view illustrating a patch for inducing twisting of skin according to an embodiment of the present disclosure, and FIGS. 9 to 10 are schematic views illustrating an operation process of a patch for inducing twisting of skin according to an embodiment of the present disclosure.

As illustrated in FIG. 8, the patch for inducing twisting of skin according to an embodiment of the present disclosure includes an attachment pad 60, a high-frequency application part 20, and a rotation part 30.

The attachment pad 60 may be closely attached to the skin, and may serve to stimulate the skin. For example, the attachment pad 60 may have a rectangular shape, and a lower surface of the attachment pad 60 may have a curved shape, a center of which is recessed, to be closely attached to the skin.

The high-frequency application part 20 may serve to apply a high frequency to the attachment pad 60. Specifically, the high-frequency application part 20 may apply a high frequency to the attachment pad 60 while the attachment pad 60 is closely attached to the skin. As a result, the high frequency applied from the high-frequency application part 20 may be delivered to the skin through the attachment pad 60 closely attached to the skin.

The rotation part 30 may serve to rotate the attachment pad 60, for example, the rotation part 30 may rotate the attachment pad 60 while the attachment pad 60 is closely attached to the skin. As a result, as the attachment pad 60 closely attached to the skin induce twisting of the skin while being rotated, the intercellular connection signals of the skin are stimulated through stretching of the skin tissues, collagen and elastic fibers of the skin are increased, and skin regeneration may be improved.

As an example, the rotation part 30 may be a driving motor. The driving motor may rotate the attachment pad 60 under the control of a processor that is manipulated by the user.

As another example, the rotation part 30 may be a knob, and the knob may manually rotate the attachment pad 60 according to the manipulation by the user.

Meanwhile, the rotation part 30 may rotate the attachment pad 60 in one direction while the attachment pad 60 is closely attached to the skin, and then may rotate the attachment pad 60 in an opposite direction. As a result, the attachment pad 60 closely attached to the skin are rotated sequentially in one direction and an opposite direction, and the twisting of the skin in one direction and the twisting of the skin in an opposite direction may be sequentially induced.

A microcomputer or a programmable logic controller (PLC) may be used as the processor (not illustrated), and may serve to control the high-frequency application part 20 and the rotation part 30. As an example, the processor may include a proximity sensor that is provided in the holder 11. The proximity sensor may measure a distance between the attachment pad 60 and the skin to identify whether the attachment pad 60 is closely attached to the skin. Furthermore, the processor may operate the high-frequency application part 20 and the rotation part 30 when it is identified that the attachment pad 60 is closely attached to the skin according to the sensing by the proximity sensor.

As illustrated in FIG. 8, the patch for inducing twisting of skin according to an embodiment of the present disclosure may further include a cylinder 40 and a pressure control part 50.

The cylinder 40 has an interior space 41, to which the attachment pad 60 is fixed, and may be closely attached to the skin. As an example, a lower surface of the cylinder 40 may be opened and an upper surface and a circumferential surface thereof may be closed, and an interior space 41 may be formed in an interior thereof. Furthermore, the attachment pad 60 may be fixed to the upper surface of the cylinder 40. Furthermore, the rotation part 30 may rotate the attachment pad 60 by rotating the cylinder 40.

The pressure control part 50 may form a negative pressure or positive pressure in the interior space 41 of the cylinder 40. Here, the negative pressure may be a pressure that is lower than the atmospheric pressure, and the positive pressure may be a pressure that is higher than the atmospheric pressure.

As an example, the pressure control part 50 may form a negative pressure in the interior space 41 of the cylinder 40 such that the attachment pad 60 is closely attached to the skin that is pulled into the interior space 41 of the cylinder 40 (see FIG. 9). Thereafter, the rotation part 30 may rotate the cylinder 40 to induce the twisting of the skin closely attached to the rotating attachment pad 60 (see FIG. 10).

The pressure control part 50 may include a pressure pipe 51 that is connected to the inner space of the cylinder 40, and a pressure regulator 52 that forms a negative pressure or positive pressure in the pressure pipe 51.

The needle tip and the patch that induce twisting of the skin according to an embodiment of the present disclosure may induce twisting of the skin while applying a high frequency to the skin to stimulate the intercellular connection signals of the skin through stretching of the skin tissues, increase collagen and elastic fibers of the skin, and improve skin regeneration.

Effects of the present disclosure are not limited to the effects mentioned above, and other effects not mentioned will be clearly understood by those skilled in the art from the following description.

According to the present disclosure, the needle tip and the patch that induce twisting of the skin according to an embodiment of the present disclosure may induce twisting of the skin while applying a high frequency to the skin to stimulate the intercellular connection signals of the skin through stretching of the skin tissues, increase collagen and elastic fibers of the skin, and improve skin regeneration.

Although an embodiment of the present disclosure is described with reference to the accompanying drawings, it will be understood by those skilled in the art to which the present disclosure pertains that the present disclosure can be carried out in other detailed forms without changing the scope and spirit or the essential features of the present disclosure. Therefore, the above-described embodiments are exemplary in all aspects, and should be construed not to be restrictive.

What is claimed is:

1. A needle tip for inducing twisting of skin, the needle tip comprising:
a tip part including a holder, and a plurality of needles inserted into the skin to form a plurality of injection holes;
a high-frequency application part configured to apply a high frequency to the plurality of needles; and
a rotation part configured to rotate the tip part.

2. The needle tip of claim 1, wherein the rotation part rotates the tip part while the plurality of needles are inserted into the skin.

3. The needle tip of claim 1, wherein the rotation part rotates the tip part after the plurality of needles are inserted into the skin.

4. The needle tip of claim 1, wherein the rotation part rotates the tip part in one direction while the plurality of needles are inserted into the skin, and then rotates the tip part in an opposite direction.

5. The needle tip of claim 1, further comprising:
a cylinder having an interior space, in which the tip part is fixed, and closely attached to the skin; and
a pressure control part configured to form a negative pressure or a positive pressure in the interior space.

6. The needle tip of claim 5, wherein the pressure control part forms the negative pressure in the interior space such that the plurality of needles are inserted into the skin being pulled into the interior space.

7. The needle tip of claim 5, wherein the pressure control part forms the positive pressure in the interior space when the plurality of needles are discharged from the plurality of injection holes to inject drug applied to the skin into the injection holes.

8. The needle tip of claim 1, wherein the high-frequency application part is configured to:
apply any one of a monopolar type high frequency and a bipolar type high frequency to the plurality of needles when the tip part is rotated in one direction; and
apply the other one of the monopolar type high frequency and the bipolar type high frequency to the plurality of needles when the tip part is rotated in an opposite direction.

9. A patch for inducing twisting of skin, the pad comprising:
an attachment pad closely attached to the skin;
a high-frequency application part configured to apply a high frequency to the attachment pad; and
a rotation part configured to rotate the attachment pad.

10. The patch of claim 9, wherein the rotation part rotates the attachment pad in one direction while the attachment pad is closely attached to the skin, and then rotates the attachment pad in an opposite direction.

11. The patch of claim 10, further comprising:
a cylinder having an interior space, in which the attachment pad is fixed, and closely attached to the skin; and
a pressure control part configured to form a negative pressure in the interior space,
wherein the pressure control part forms the negative pressure in the interior space such that the attachment pad is closely attached to the skin being pulled into the interior space.
